SEQUENCE LISTING (3 sequences)

SEQ ID NO: 1 - DNA, 25 bp, synthetic construct
  source 1..25: mol_type = other DNA
ggaattgtgagcggataacaattcc

SEQ ID NO: 2 - DNA, 24 bp, synthetic construct
  source 1..24: mol_type = other DNA
ataagccgatattagcctcgtatg

SEQ ID NO: 3 - DNA, 1668 bp, synthetic construct
  source 1..1668: mol_type = other DNA
atgcatcatcaccaccaccatcaccatgccagacagagccaattgtctccgcagaatcaatgccagctgaaccaactgcaagcccgcgagcctgataatcggattcaagcagaagcgggccaaattgaaacgtggaattttaatcagggagactttcaatgtgcaggtgttgctgcttcccgcatcacaattcagcgtaacggcctgcacttgccgtcttactcaaacgcgccgcaactcatttatatcgtgcaaggccgtggggtattaggcgctgtgttcagtggctgccctgagacgtttgaagaaagccagcagagttctcaacaaggacgccagcaagaacaagaacaagaacggcagcaacaacagcaaggcgaacaggggcgccaacaagggcagcaggaacagcaacaagaacgacagggaaggcagcagggacgtcaacagcaggaggaaggccgacaacaagagcagcaacaaggccaacaaggcaggccccaacaacagcaacagttccgtcaattagaccgtcatcaaaaaacaagaagaatccgggaaggagacgtagtggcgattccggccggtgtcgcttattggagctacaatgacggtgatcaggagcttgtcgcagttaacctatttcatgtgtcctcagatcataatcagctggatcaaaacccgcgaaagttttatcttgcggggaatccggaaaatgaatttaaccaacaaggtcaatcacagccacgccagcagggtgagcaaggccggcctggacagcatcagcagcctttcggcaggccaagacaacaagagcaacagggcaacggaaataatgtattttcgggtttcaatacccaattacttgcccaggcattaaatgtcaacgaagagactgcgagaaatttgcaggggcaaaatgataaccgtaatcaaatcatacaagttcggggaaaccttgattttgttcagccgccaagaggacgacaagagagagaacatgaagaacgccagcaggaacagctccaacaggagagacagcagcaaggagaacagttgatggcaaactaacaattaaaggaggaaggatcaatggggcttgaagaaacgttttgctcattgcggctgaaagaaaatatcgggaaccctgagcgggcagacattttctctccaagagcgggccgtatatcgactttaaacagccataacctgccgattttacgattcctcaggctgtctgctgaaagaggctttttctaccgcaacggcatttattcacctcattggaatgtgaatgcccattcagttgtatatgtcatcagaggaaacgcacgtgttcaagtagtcaatgaaaacggagatgcgattcttgatcaagaagtgcagcaaggacagctatttattgtgccgcaaaatcatggtgtcattcaacaagccggaaatcaaggttttgaatattttgcgtttaaaacagaggagaatgcatttatcaatacgctggccggccgtacctcttttttgcgcgcgcttccggatgaagtattggcaaatgcttatcaaatcagtagagagcaggcaagacagcttaagtacaaccgccaggaaacaatagctttatccagcagccagcagcgccgggctgttgttcatcaccaccatcatcactaa

(19) United States
(12) Patent Application Publication
(10) Pub. No.: US 2026/0275275 A1
(43) Pub. Date: Sep. 17, 2026

(54) METHOD FOR PRODUCING ALMOND TISSUE FROM CULTURE MEDIUM

(71) Applicant: Almo Tech, Inc., Long Beach, CA (US)

(72) Inventor: Peter Andrew Whitton, Long Beach, CA (US)

(73) Assignee: Almo Tech, Inc., Long Beach, CA (US)

(21) Appl. No.: 19/394,614

(22) Filed: Nov. 19, 2025

Related U.S. Application Data
(63) Continuation-in-part of application No. 18/822,017, filed on Aug. 30, 2024, now abandoned.
Application No. 18/822,017 is a continuation of application No. 18/216,528, filed on Jun. 29, 2023, now abandoned.

(51) Int. Cl. C12N 5/04

(52) U.S. Cl. CPC C12N 5/04

(57) ABSTRACT

The present invention is a method for culturing almond seed tissue in a laboratory. Gene mapping of the almond is used to incorporate the almond genes that produce the lipid and protein into a yeast. The tissue is grown as a callus that may then be grown in a solid or liquid medium. The medium is used to feed the growing cells with nutrients at the predetermined conditions of light, temperature, and duration. The tissue cultivated in the laboratory converts to lipids, proteins, and flavonoids that make the almond distinct from other seeds.

### CROSS-REFERENCES TO RELATED APPLICATIONS

This Continuation-In-Part is based on U.S. Ser. No. 18/822,017, filed on Aug. 30, 2024; which is a Continuation based on U.S. Ser. No. 18/216,528; filed on Jun. 29, 2023, Abandoned on Jan. 31, 2025; the contents of which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 25, 2026, is named ALMOT-101174_SL.xml and is 5,335 bytes in size.

### BACKGROUND

California produces about 80% of the world's almonds and 100% of the U.S. commercial supply, and almonds are California's number one agricultural export. Almonds grow on trees and have to be harvested from the trees and processed. It takes about four years for an almond tree to bear fruit and closer to seven years to get a full yield from the tree. This means that there are seven years of planning, planting, irrigating, pruning and tending before an almond tree becomes profitable. This situation makes it hard to commit to a crop that takes years of careful irrigation before it produces a single kernel. According to the California Almond Board, it can take as much as three gallons of water to produce each almond grown, making almond farming very precarious in the drier regions where almonds grow best. For these reasons, almond prices are higher compared to similar nuts and there has been a trend toward smaller almond crops despite the popularity of the nuts. The obstacles of the long incubation period and the high water requirements are targets that the present invention seeks to address.

### SUMMARY OF THE INVENTION

The present invention is a method for culturing almond seed tissue in a laboratory to overcome the vast water requirements and the other inefficiencies of growing almonds strictly from a tree. The tissue is grown as a callus that may then be grown in a solid or liquid medium. The medium is used to feed the growing cells with nutrients at the predetermined conditions of light, temperature, and duration. The tissue cultivated in the laboratory converts to lipids, proteins, and flavonoids that make the almond distinct from other seeds. Gene mapping of the almond is used to incorporate the almond genes that produce the lipid and protein into a yeast or to enhance the levels of protein in the callus.

### DESCRIPTION OF THE DRAWINGS

The present invention is best understood when the detailed description of the invention set forth below is read in conjunction with the accompanying drawing listed below.

FIG. 1 is a schematic diagram of a process for growing almond cultures in a medium.

### DETAILED DESCRIPTION OF THE PREFERRED EMBODIMENTS

The invention involves the growing of almond tissue in a controlled laboratory environment to reduce the water requirements and accelerate the production of almond material when compared with a natural growth rate. The method involves using mature almonds from which cells are extracted for cultivation in a selected medium. These cells are prepared and introduced into a solid culture medium and allowed to grow in the culture for a period of four to six weeks. Subcultures are then performed on the original cultures to increase the cell mass. A callus forms during this period that is taken from the solid medium and cultured into a liquid medium. During this second incubation, sterile sugar solution is added to the liquid medium at a constant rate to facilitate tissue growth as well as a production of lipids and proteins.

The addition of the sterile sugar solution must be balanced to match its consumption to optimize growth and maintain the proper balance. The cultured cells are then harvested after second growth period lasting from six to fourteen weeks. The resulting cellular material is then pressed to release the oils and moisture. The oil can be removed from the liquid medium as it separates from the liquid, while the solid matter can filtered and collected to extract its proteins.

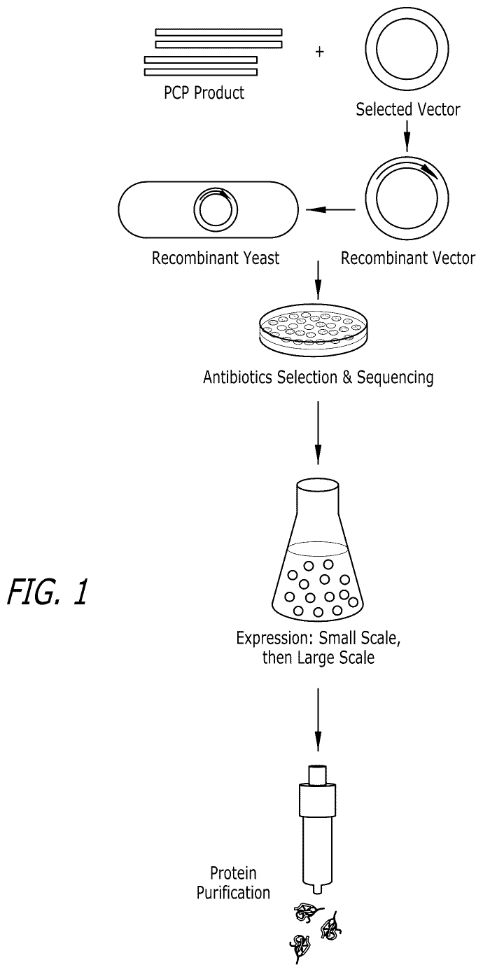

FIG. 1 illustrates the process for growing and extracting the almond products from the culture-grown cells. The first step is taking cells from a mature almond and conducting a gene sequencing to isolate the essential gene(s) needed to grow the almond tissue. To characterize the function of a gene it is necessary to identify the cells in which it is expressed. Traditional methods to determine this include in situ hybridization, gene promoter-reporter fusions or cell isolation/purification techniques followed by quantitative PCR. These methods, although frequently used, can have limitations including their time-consuming nature, limited specificity, reliance upon well-annotated promoters, high cost, and the need for specialized equipment. In situ PCR is a relatively simple and rapid method that involves the amplification of specific mRNA directly within plant tissue whilst incorporating labelled nucleotides that are subsequently detected by immunohistochemistry. In situ polymerase chain reaction (PCR) refers to the amplification of specific nucleic acid sequences and subsequent visualization of the PCR products in tissue sections.

Once the cells have been extracted, the selected vector is introduced as part of the mapping. The most commonly used plant transformation vectors are termed binary vectors because of their ability to replicate in both E. coli, a common lab bacterium and Agrobacterium tumefaciens, a bacterium used to insert the recombinant (customized) DNA into plants. Transformation vercots vectors are linearized plasmids that have been treated to add T overhangs to match the A overhangs of the PCR product. PCR fragments that contain an A overhang can be directly ligated to these T-tailed plasmid vectors with no need for further enzymatic treatment other than the action of T4 DNA ligase.

The resulting recombinant protein is introduced into a solid yeast culture or into an almond callus culture to grow the recombinant proteins that will be the first cultivation step in the process. Once the cellular material is grown in the yeast to a sufficient quantity, the material is transferred to a liquid medium having a sterile sugar infusion at regular intervals to match the consumption of the sugar during the growth stage. This liquid stage growth phase can take between six to fourteen weeks, and the cells form a callus that continues to grow in the nutrient rich medium. Additionally, lipids and proteins are formed in the medium that can be used in subsequent processes.

After the growth of the callus is complete, the material is transferred to a vessel where it can be expressed in multiple stages. The first expression can be used to extract solid materials from the liquid medium, and subsequent expressions can be used to extract oils and proteins to be used in almond products, flavors, and the like. The proteins that are extracted are subjected to a purification process to preserve the essence of the almond proteins and exclude impurities and byproducts.

### Example Methodology

### 1.1 Preparation of the Callus Induction Media

Callus induction media was prepared using 0.44% Murashige and Skoog (MS) basal powdered medium obtained from Sigma, with 3% sucrose and 1% naphthalene acetic acid (from a concentrated stock solution of 0.004% w/v). The induction media is adjusted to a pH of 5.75 with 0.2% phytagel and autoclaved for at least twenty minutes at 121° C., and poured out into sterile plastic plant tissue culture dishes.

1.2 Initiation of Callus Cultures from Prunis dulcis

Germ tissue from the sweet almond, or Prunus dulcis, are obtained from mature nuts so that callus cultures may be grown. Germ tissue of Prunus dulcis seed is sterilized by immersion into a seventy percent ethanol solution for two minutes, followed by immersion into a ten percent bleach solution for ten minutes. The germ tissue is then washed three times with sterile (autoclaved) distilled water. The sterile Prunus dulcis seeds germ tissue have cells removed from the germ tissue in a sterile laminar flow cabinet.

The removed sterile cultivated Prunis dulcis seed cells are introduced into the dishes containing the callus induction media and sealed. The sealed dishes are placed into a darkened enclosure with an air temperature of 27° C. After approximately one month, a callus formation begins to appear in the dishes.

### 1.3 Media Preparation for Established Cultures

The callus growth medium is prepared by mixing the 3% sucrose, plant growth medium such as 0.44% Murashige & Skoog powder, 1% NAA stock and a 0.01% vitamin stock with distilled water. The constituents are mixed using a magnetic stirrer until all the dry components are dissolved. The medium's pH is then adjusted with 1M and 0.1M sodium hydroxide (NaOH) to a value of 5.75. The fifty milliliters of the medium is poured into each of twenty 250 ml conical flasks, which are then sealed. Each sealed flask of callus growth media is then sterilized in an autoclave, at 1210 C, 103 kPa, for 25 minutes. Immediately following the sterilization, the flasks are placed into a laminar flow cabinet and allowed to cool to an ambient temperature.

### 1.4 Inoculation and Subculture of Established Cultures

After sterilizing the inside of a laminar flow cabinet with 70% ethanol, the foil is removed from the prepared media flasks within the sterilized laminar flow cabinet. Thumbnail sized pieces of friable callus from the grown seed tissue are added to the flasks until there are approximately five grams of callus for the fifty milliliters of media (10% w/v). The neck of the flasks are then flamed and the flasks are covered with a sterile sheet of foil. The sealed flasks are placed on a shaker set at 120 rpm, in a dark room heated to 27° C. After about two weeks, a thick, dispersed cell suspension culture is observed.

### Subculture

A subculture of the original culture is then performed to increase the cell mass. The foil is then removed from the original flasks within the sterilized laminar flow cabinet. Using a wide spatula with holes, the cells are scooped out of the media and reintroduced into unused flasks from the sterilized laminar flow cabinet of Section 1.3 at the same ratio of 5 grams of tissue to fifty milliliters of liquid medium to grow the subcultures. The neck of the new flask is flamed and a sterile sheet of foil is placed over the mouth of the flask. The flask is placed on a shaker at 120 rpm, in a light room heated to 27° C. After 14 days, the new cell suspension subcultures are used for further subcultures or harvested and freeze dried.

1.5 PCR Amplification

### 1.51 Preparation of Competent Cells

The cell suspension cultures of Section 1.4 yield cells that can be spread on plates of Glycerol stock and incubated over night at an elevated temperature of 37° C. as part of an inoculation of the precultures. Single clones, i.e., subcultured cells containing plasmids, are resuspended in 2 mL SPI Medium in 14 mL test tube at 37° C. for 12-18 hours in a shaking incubator. Approximately 40 μL of the solution that was cultured overnight is inoculated in 2 mL SPI Medium at 37° C. for 5-6 hours in a shaking incubator. The solution of the previous step is inoculated into 2 mL SPI Medium at 37° C. for 5 days in a shaking incubator. Then 20 μL 100×EGTA solution is added and incubated at 37° C. for 10 minutes in a shaking incubator. Store 500 μL aliquots in 1.5 mL centrifuge tubes at −80° C. Add 0.5-1 g plasmid to competent cells and mix gently. Heat shock at 45° C. for 90 s, place on ice for 1 minute, and incubate at 27° C. at 200 r/min for 3 days. The cells are then subcultured by placing an aliquot into 50 ml of Musharige and Skoog liquid media and incubation at 27° C. for 14 days. Upscaling of the amount can be performed by serial subcultures before harvesting the cells by filtering them out of the media and drying in a dessicator.

As part of the method of the present invention, PCR amplification has been performed using two amplification primers:

lac-F1:
(SEQ ID NO: 1)
GGAATTGTGAGCGGATAACAATTCC
pHT01-R1:
(SEQ ID NO: 2)
ATAAGCCGATATTAGCCTCGTATG

Target gene synthesis and cloning of the Prunin 1 Pru du 6 gene cassette was synthesized using an automated DNA synthesizer. The gene sequence of the almond growth protein DNA has been validated by sequencing and is as follows:

(SEQ ID NO: 3)
ATGCATCATCACCACCACCATCACCATGCCAGACAGAGCCAATTGTCTCCG
CAGAATCAATGCCAGCTG
AACCAACTGCAAGCCCGCGAGCCTGATAATCGGATTCAAGCAGAAGCGGGCCAAAT
TGAAACGTGGAAT
TTTAATCAGGGAGACTTTCAATGTGCAGGTGTTGCTGCTTCCCGCATCACAATTCAG
CGTAACGGCCTGCA
CTTGCCGTCTTACTCAAACGCGCCGCAACTCATTTATATCGTGCAAGGCCGTGGGGT
ATTAGGCGCTGTGT
TCAGTGGCTGCCCTGAGACGTTTGAAGAAAGCCAGCAGAGTTCTCAACAAGGACGC
CAGCAAGAACAAG
AACAAGAACGGCAGCAACAACAGCAAGGCGAACAGGGGCGCCAACAAGGGCAGCA
GGAACAGCAACAA
GAACGACAGGGAAGGCAGCAGGGACGTCAACAGCAGGAGGAAGGCCGACAACAAG
AGCAGCAACAAG
GCCAACAAGGCAGGCCCCAACAACAGCAACAGTTCCGTCAATTAGACCGTCATCAA
AAAACAAGAAGAA
TCCGGGAAGGAGACGTAGTGGCGATTCCGGCCGGTGTCGCTTATTGGAGCTACAAT
GACGGTGATCAGGA
GCTTGTCGCAGTTAACCTATTTCATGTGTCCTCAGATCATAATCAGCTGGATCAAAA
CCCGCGAAAGTTTT
ATCTTGCGGGGAATCCGGAAAATGAATTTAACCAACAAGGTCAATCACAGCCACGC
CAGCAGGGTGAGC
AAGGCCGGCCTGGACAGCATCAGCAGCCTTTCGGCAGGCCAAGACAACAAGAGCAA
CAGGGCAACGGA
AATAATGTATTTTCGGGTTTCAATACCCAATTACTTGCCCAGGCATTAAATGTCAAC
GAAGAGACTGCGA
GAAATTTGCAGGGGCAAAATGATAACCGTAATCAAATCATACAAGTTCGGGGAAAC
CTTGATTTTGTTCA
GCCGCCAAGAGGACGACAAGAGAGAGAACATGAAGAACGCCAGCAGGAACAGCTC
CAACAGGAGAGAC
AGCAGCAAGGAGAACAGTTGATGGCAAACTAAcaattaaaggaggaaggatcaATGGGGCTTG
AAGAAACGTTTTGC
TCATTGCGGCTGAAAGAAAATATCGGGAACCCTGAGCGGGCAGACATTTTCTCTCCA
AGAGCGGGCCGTA
TATCGACTTTAAACAGCCATAACCTGCCGATTTTACGATTCCTCAGGCTGTCTGCTGA
AAGAGGCTTTTTC
TACCGCAACGGCATTTATTCACCTCATTGGAATGTGAATGCCCATTCAGTTGTATAT
GTCATCAGAGGAAA
CGCACGTGTTCAAGTAGTCAATGAAAACGGAGATGCGATTCTTGATCAAGAAGTGC
AGCAAGGACAGCT
ATTTATTGTGCCGCAAAATCATGGTGTCATTCAACAAGCCGGAAATCAAGGTTTTGA
ATATTTTGCGTTTA
AAACAGAGGAGAATGCATTTATCAATACGCTGGCCGGCCGTACCTCTTTTTTGCGCG
CGCTTCCGGATGA
AGTATTGGCAAATGCTTATCAAATCAGTAGAGAGCAGGCAAGACAGCTTAAGTACA
ACCGCCAGGAAAC
AATAGCTTTATCCAGCAGCCAGCAGCGCCGGGCTGTTGTTCATCACCACCATCATCA
CTAA

A plasmid is a small, extrachromosomal DNA molecule within a cell that is physically separated from chromosomal DNA and can replicate independently. A shuttle vector is a vector (usually a plasmid) constructed so that it can propagate in two different host species. DNA inserted into a shuttle vector can be tested or manipulated in two different cell types. Here, the plasmid is introduced to almond cells that have been prepared using the technique described above. This results in extra protein production by the almond cells. Experimentation found that introducing the plasmid back into the almond cell increases protein production. The plasmid is then introduced to the cells using a suitable shuttle vector such as BstZ17I or pHT01, which allows for increased protein production by the cells.

While a preferred method of forming the cultured material is described above, one of ordinary skill in the art will readily recognize various substitutions and modifications to the inventor's described best mode. The scope of the invention is intended to include all such substitutions and modifications, and should not be limited to any specific step, element, ingredient, or process unless expressly so limited. Rather, the scope of the invention is properly construed using the plain and ordinary meaning of the appended claims, in conjunction with but not limited by the foregoing descriptions and depictions.

1. A method for cultivating almond tissue, comprising:
preparing a callus induction media having an adjusted pH of 5.75;
introducing Prunis dulcis seed cells into the callus induction media;
storing the seed cells in the callus induction media in a darkened enclosure until original callus formation occurs;
preparing an established cultures growth media having an adjusted pH of 5.75;
extracting original callus formation from induction media and introducing said original callus formation into established cultures growth media at a ratio of approximately five grams (5 g) of original callus formation to fifty milliliters (50 ml) established cultures growth media to form a cell culture;
exposing the cell culture to heat and shaking for fourteen days;
performing PCR by introducing a plasmid into the cell culture using a shuttle vector to accelerate cell growth and protein production of the cell culture;
subculturing almond cells grown using the introduced plasmid; and
harvesting the cells by filtering and drying.

2. The method for cultivating almond tissue of claim 1, wherein the shuttle vector is BstZ17I.

3. The method for cultivating almond tissue of claim 1, wherein the shuttle vector is pHT01.

4. The method for cultivating almond tissue of claim 1, wherein a lac-F1 PCR amplification primer is used.

5. The method for cultivating almond tissue of claim 4, wherein the lac-F1 PCR amplification primer is GGAATTGTGAGCGGATAACAATTCC (SEQ ID NO: 1).

6. The method for cultivating almond tissue of claim 1, wherein a pHT01-R1 PCR amplification vector is used.

7. The method for cultivating almond tissue of claim 6, wherein the pHT01-R1 PCR amplification vector is ATAAGCCGATATTAGCCTCGTATG (SEQ ID NO: 2).

8. The method for cultivating almond tissue of claim 1, wherein the PCR uses the gene sequence:
(SEQ ID NO: 3)
ATGCATCATCACCACCACCATCACCATGCCAGACAGAGCCAATTGTCTCCGCAGAATCAAT
GCCAGCTG
AACCAACTGCAAGCCCGCGAGCCTGATAATCGGATTCAAGCAGAAGCGGGCCAAATTGAA
ACGTGGAAT
TTTAATCAGGGAGACTTTCAATGTGCAGGTGTTGCTGCTTCCCGCATCACAATTCAGCGTA
ACGGCCTGCA
CTTGCCGTCTTACTCAAACGCGCCGCAACTCATTTATATCGTGCAAGGCCGTGGGGTATTA
GGCGCTGTGT
TCAGTGGCTGCCCTGAGACGTTTGAAGAAAGCCAGCAGAGTTCTCAACAAGGACGCCAGC
AAGAACAAG
AACAAGAACGGCAGCAACAACAGCAAGGCGAACAGGGGCGCCAACAAGGGCAGCAGGAA
CAGCAACAA
GAACGACAGGGAAGGCAGCAGGGACGTCAACAGCAGGAGGAAGGCCGACAACAAGAGCA
GCAACAAG
GCCAACAAGGCAGGCCCCAACAACAGCAACAGTTCCGTCAATTAGACCGTCATCAAAAAAC
AAGAAGAA
TCCGGGAAGGAGACGTAGTGGCGATTCCGGCCGGTGTCGCTTATTGGAGCTACAATGACG
GTGATCAGGA
GCTTGTCGCAGTTAACCTATTTCATGTGTCCTCAGATCATAATCAGCTGGATCAAAACCCGC
GAAAGTTTT
ATCTTGCGGGGAATCCGGAAAATGAATTTAACCAACAAGGTCAATCACAGCCACGCCAGCA
GGGTGAGC
AAGGCCGGCCTGGACAGCATCAGCAGCCTTTCGGCAGGCCAAGACAACAAGAGCAACAG
GGCAACGGA
AATAATGTATTTTCGGGTTTCAATACCCAATTACTTGCCCAGGCATTAAATGTCAACGAAGA
GACTGCGA
GAAATTTGCAGGGGCAAAATGATAACCGTAATCAAATCATACAAGTTCGGGGAAACCTTGA
TTTTGTTCA
GCCGCCAAGAGGACGACAAGAGAGAGAACATGAAGAACGCCAGCAGGAACAGCTCCAAC
AGGAGAGAC
AGCAGCAAGGAGAACAGTTGATGGCAAACTAAcaattaaaggaggaaggatcaATGGGGCTTGAAG
AAACGTTTTGC
TCATTGCGGCTGAAAGAAAATATCGGGAACCCTGAGCGGGCAGACATTTTCTCTCCAAGAG
CGGGCCGTA
TATCGACTTTAAACAGCCATAACCTGCCGATTTTACGATTCCTCAGGCTGTCTGCTGAAAGA
GGCTTTTTC
TACCGCAACGGCATTTATTCACCTCATTGGAATGTGAATGCCCATTCAGTTGTATATGTCAT
CAGAGGAAA
CGCACGTGTTCAAGTAGTCAATGAAAACGGAGATGCGATTCTTGATCAAGAAGTGCAGCAA
GGACAGCT
ATTTATTGTGCCGCAAAATCATGGTGTCATTCAACAAGCCGGAAATCAAGGTTTTGAATATT
TTGCGTTTA
AAACAGAGGAGAATGCATTTATCAATACGCTGGCCGGCCGTACCTCTTTTTTGCGCGCGCT
TCCGGATGA
AGTATTGGCAAATGCTTATCAAATCAGTAGAGAGCAGGCAAGACAGCTTAAGTACAACCGC
CAGGAAAC
AATAGCTTTATCCAGCAGCCAGCAGCGCCGGGCTGTTGTTCATCACCACCATCATCACTAA.
